(19) United States
(12) Patent Application Publication
(10) Pub. No.: US 2020/0046552 A1
(43) Pub. Date: Feb. 13, 2020

(54) CRYOTHERAPIES

(71) Applicant: MIRAKI INNOVATION THINK TANK LLC, Cambridge, MA (US)

(72) Inventors: Christopher J.P. Velis, Lexington, MA (US); Karen Miller, South Dartmouth, MA (US); Tarik S. Chaudhry, Cambridge, MA (US); Susan Arias, Cambridge, MA (US)

(21) Appl. No.: 16/490,244

(22) PCT Filed: Mar. 1, 2018
(86) PCT No.: PCT/US2018/020387

§ 371 Date: Aug. 30, 2019

Related U.S. Application Data
(60) Provisional application No. 62/465,336, filed on Mar. 1, 2017.

(51) Int. Cl. A61F 7/12

(52) U.S. Cl. CPC A61F 7/12; A61F 2007/0063

(57) ABSTRACT

Delivering cold slurry to internal tissues can be used in therapies for treating a variety or medical diseases and conditions. For example, cold slurry can activate brown adipose tissue to treat obesity. Cold slurry can be delivered to tissue injured by trauma or disease to reduce inflammation, which in turn reduces pain and leads to quicker recovery. Cold slurry therapy can also be used to treat muscular and neurological disorders as well as to manage pain. The cooling effect of cold slurry delivered at or near a nerve reduces innervation that nerve, which in turn reduces spasms or pain. The cooling effect can also cause tissue to undergo the cell death. Advantageously, cold slurry therapies can be used to reduce or remove tissue, for example, to treat fibroadenomas or scar tissue.

### BACKGROUND

Use of cryotherapy or the application of cold for the treatment of an injury or disease is an established method for treating acute soft tissue injuries as well as for reducing recovery time after injury and surgery. Cryotherapy can maintain physiological and biological effects in various tissues as well as in neurological action regarding sensory and motor nerves, and physiologic inflammatory reactions. Other uses of cryotherapy include fat reduction, ablation, and amputation as well as preservation of tissues in surgical settings.

In the area of fat reduction, cryotherapy can be used to stimulate the burning of calories. Non-shivering thermogenesis is the metabolic process of producing heat from substances, such as free fatty acids, which does not involve shivering. Recent studies have shown that this process primarily takes place in brown adipose tissue and is controlled by the activity of sympathetic nerve supply. In humans, when fully stimulated, brown adipose tissue can generate three hundred times more heat than any other tissue in the body. Approximately two ounces of brown adipose tissue can burn 300 to 500 calories a day—enough to lose up to one pound in a week. As such, there is great interest in activating brown adipose tissue as a way for treating obesity and weight-related disorders.

Exposing the body to cold temperatures (e.g., 16° C. or below) is known to stimulate brown adipose tissue activity. Prior approaches to cold-inducing thermogenesis include sitting in a cold room or being immersed in a cold water bath for several hours, another is to wrap a cooling blanket around the body. These approaches are extremely uncomfortable because they involve cooling the entire body or a large portion of the body. Yet another approach is to implant a cooling device into the body to cool the inside. That approach is lacking because it requires leaving a foreign object inside the body where it might be rejected or breakdown.

### SUMMARY

Delivering a cold slurry to tissues inside a patient can be used in a number of therapies for treating a variety or medical diseases and conditions. For example, cold slurry can be delivered at or near a patient's adipose tissue, colonic tissue, abdominal tissue or hypothalamic tissue. The cooling effect of the cold slurry on these tissues can stimulate thermogenesis in the patient's brown adipose tissue and increase general metabolic activity. As such, this cold slurry therapy can be used to treat obesity or a weight-related disorder. In another example, cold slurry can be delivered at or near an internal tissue injured by trauma or disease. The cooling effect of the cold slurry on the injured tissue can reduce inflammation, which in turn can reduce pain and lead to a quicker recovery.

Cold slurry therapy can also be used to treat a number of muscular and neurological disorders as well as to treat pain. The cooling effect of a cold slurry delivered at or near a nerve can reduce the innervation or conduction of that nerve. This in turn reduces spasms or pain. The cooling effect of a cold slurry on tissue can also cause the tissue to undergo the cell death. Advantageously, cold slurry therapies can be used to reduce or remove tissue, for example, to treat fibroadenomas or scar tissue.

### BRIEF DESCRIPTION OF DRAWINGS

FIG. 1 is an outline of an adult human body showing the location of brown adipose tissue depots.

FIG. 2 is a diagram of an example procedure for cooling a subject's internal tissue with a cold slurry to induce thermogenesis in brown adipose tissue.

FIG. 3 is a diagram of an example procedure for cooling a subject's abdominal tissue with a cold slurry to induce thermogenesis in brown adipose tissue.

FIG. 4 is a diagram of an example procedure for cooling a subject's colonic tissue with a cold slurry to induce thermogenesis in brown adipose tissue.

FIG. 5 is a diagram of an example procedure for cooling a subject's hypothalamic tissue with a cold slurry to induce thermogenesis in brown adipose tissue.

FIG. 6 is a diagram of an example procedure for cooling a subject's nerve with a cold slurry to mediate a neurological action by the nerve or to remove the nerve.

### DETAILED DESCRIPTION

The present invention involves cold slurry therapies including delivering a cold slurry to tissue inside a subject to induce thermogenesis in the subject's brown adipose tissue. When delivered to the internal tissue, the cold slurry cools the internal tissue and, in some instances, the surrounding tissue as well. Exposure to cold temperature signals the sympathetic nervous system, and triggers the release of catecholamine neurotransmitters, e.g., norepinephrine, that stimulates β-adrenergic receptors, initiating a cascade of intracellular events in brown adipose tissue and resulting in activation of the mitochondrial uncoupling protein 1 (UCP-1). UCP-1 is located in the inner mitochondrial membrane and serves to uncouple oxidative phosphorylation by promoting a proton leak across the mitochondrial membrane, thereby generating heat and lowering ATP synthesis. As a result, calories are burned in the process of non-shivering thermogenesis.

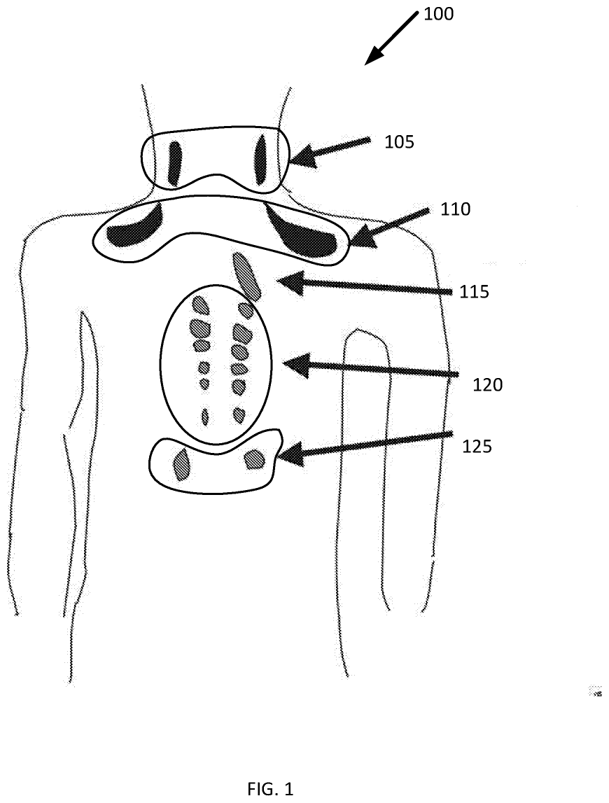

FIG. 1 shows where depots of brown adipose tissue can be found in an adult human body 100. The neck depots 105 are located on both sides of the neck. The supraclavicular depots 110 are located between the shoulder blades. The mediastinum (para-aortic) depot 115 is located near the heart. The paravertebral depots 120 are located along the spinal cord. The suprarenal depots 125 surround the kidneys.

Thermoreceptors located in surface and core body parts detect temperature and transmit temperature information to the pen-optic area (POA) of the hypothalamus, where the sense of hot or cold is perceived. Thermoreceptors located within the body or “core thermoreceptors” include the POA itself, which contains neurons whose activity is affected by local brain temperature. Temperature changes in the spinal cord can also affect the activity of thermoregulatory neurons in the POA. Splanchnic and vagus nerve afferent fibers distributed in the abdomen and exhibit responses to temperature changes similar to those of thermoreceptors located on the body surface. Thermoreceptors have also been identified in different vagal territories, including the gastrointestinal and respiratory tracts.

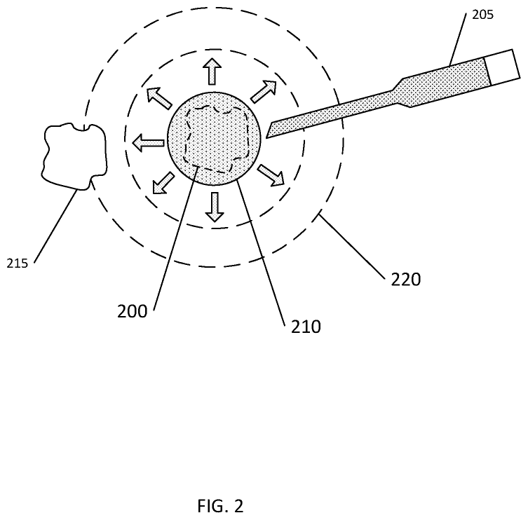

FIG. 2 shows internal tissue 200 (in phantom line) located inside a subject. The internal tissue 200 can be adipose tissue located in the subject's belly, for example. Cold slurry can be delivered to the internal tissue 200 from a delivery device 205 that is located outside the subject's body. (The delivery device 205 and cold slurry are described in greater detail at the end this disclosure.)

The cold slurry, shown in the figure as delivered cold slurry 210, can induce non-shivering thermogenesis in the subject's brown adipose tissue by way of the sympathetic control mechanism described above. For example, the affected tissue 200 includes a cold thermoreceptor that senses coldness. Upon sensing the coldness of the delivered cold slurry 210, the cold thermoreceptor sends a signal via the sympathetic pathway to the subject's hypothalamus. The hypothalamus in turn stimulates brown adipose tissue leading to the non-shivering thermogenesis.

The cold thermoreceptor can also be located in adjacent tissue 215 near the delivered cold slurry 210. After delivery, the affected area 220 expands to a size larger than the initial delivery site (shown in the figure as arrows radiating outwardly from the delivered cold slurry 210 and dashed circles of increasing size). The affected area 220 reaches a size encompassing a portion of the adjacent tissue 215 and the coldness of the delivered cold slurry 210 can be sensed by the cold thermoreceptor, thus triggering non-shivering thermogenesis in the subject's brown adipose tissue.

The cooling effect of the delivered cold slurry 210 is localized to tissue being treated (i.e., internal tissue 200) and surrounding tissue (i.e., adjacent tissue 215). In this way, any discomfort caused by the cold treatment is limited. The cold slurry is sterile and biocompatible; and, as such, the delivered cold slurry 210 can be advantageously left in the body (e.g. no removal of the slurry is necessary after cooling has been effected). The cold slurry can be delivered to other internal tissues, such as abdominal tissue, colonic tissue, and hypothalamic tissue as described below.

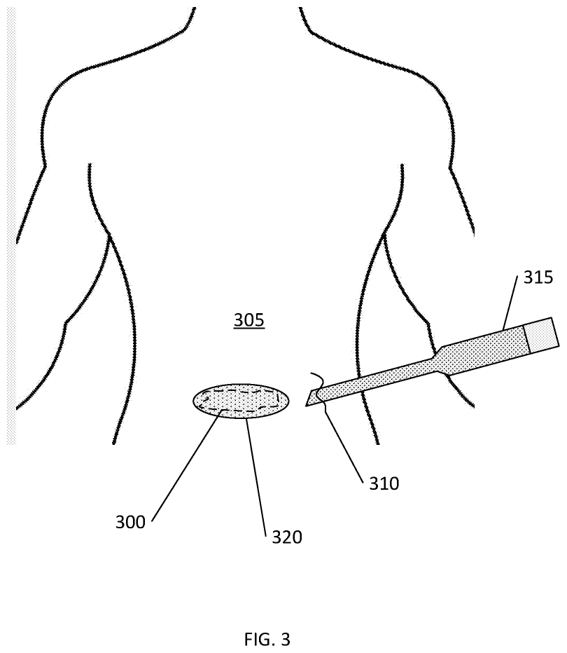

FIG. 3 shows an example procedure for cooling a subject's abdominal tissue 300 (shown with phantom lines) with a cold slurry to induce thermogenesis in the subject's brown adipose tissue. An area around a subject's abdomen 305 is cleaned and an entry point 310 is marked on the skin underlying the abdominal tissue 300.

In this example, the cold slurry is delivered to the abdominal tissue 300 using a syringe 315. The syringe 315 is inserted into the entry point 310 and advanced to the abdominal tissue 300 (or to tissue near the abdominal tissue 300). The cold slurry is then injected at (or near) the abdominal tissue 300, shown in the figure as delivered cold slurry 320. The delivered cold slurry 320 cools the abdominal tissue 300 directly (or indirectly). Cold thermoreceptors in the abdominal tissue 300 sense the cold and stimulate non-shivering thermogenesis in the subject's brown adipose tissue.

An amount of cold slurry can be delivered to multiple sites at (or near) the abdominal tissue 300. Beneficially, this increases the amount of abdominal tissue 300 that is exposed to the cold slurry and cooled, and can improve the effectiveness of the treatment.

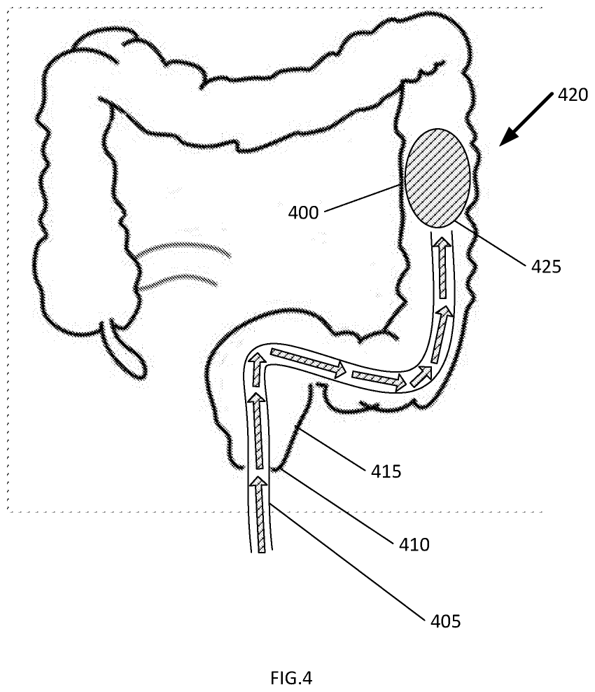

FIG. 4 shows an example procedure for cooling a subject's colonic tissue 400 with a cold slurry to induce thermogenesis in the subject's brown adipose tissue. A catheter 405 is inserted into the subject's anus 410 and advanced through the rectum 415 until it reaches the colon 420. Cold slurry is pumped (e.g., using a syringe) from outside the subject through the catheter 405 (shown in the figure as a series of arrows) and delivered to the colonic tissue 400. The cold slurry, shown in the figure as delivered cold slurry 425, cools the colonic tissue 400. Cold thermoreceptors in the colonic tissue 400 sense the coldness, which in turn stimulates non-shivering thermogenesis in the subject's brown adipose tissue.

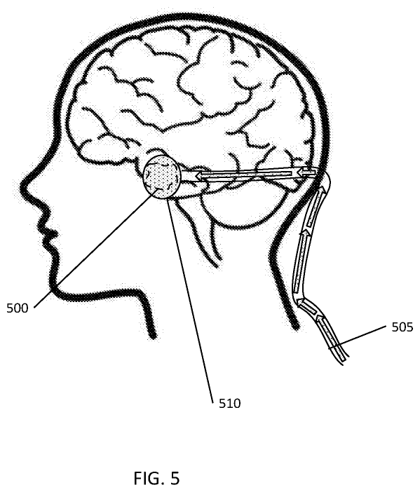

FIG. 5 shows an example procedure for cooling a subject's hypothalamic tissue 500 (shown with phantom lines) with a cold slurry to induce thermogenesis in the subject's brown adipose tissue. A port (not shown) is implanted through the subject's skin and skull bone overlying the hypothalamic tissue 500. Outside of the subject's body, a tube 505 (having a single lumen or multiple lumens) is connected to the port. A syringe or pump (not shown) connected to the other end of the tube 505 delivers the cold slurry through the tube 505 (shown in the figure as a series of arrows) to the hypothalamic tissue 500. The cold slurry, shown in the figure as delivered cold slurry 510, cools the hypothalamic tissue 500. Cold thermoreceptors in the hypothalamic tissue 500 sense the coldness, which in turn stimulates non-shivering thermogenesis in the subject's brown adipose tissue. This arrangement can be used for multiple delivers of cold slurry over a period a time. This is particularly advantageous for a long term administration of cold slurry.

The aforementioned procedures are useful for treating obesity and weight-related disorders. Generally, treatment methods include administering an effective amount of cold slurry (as described above) to a subject in need of treatment, including a subject that has been diagnosed to be in need of such treatment.

The treatment methods can include identifying a subject in need of treatment (e.g., a subject having, or at risk of having, obesity or developing a weight-related disorder), and administering to the subject an effective amount of cold slurry (as described above). In a convenient example, the subject is diagnosed as being an overweight or obese subject (e.g., having a body mass index (BMI) of 25-29 or 30 or above) or a subject with a weight-related disorder. A subject in need of treatment can be selected based on the subject's body weight or BMI.

In some examples of the treatment method, subject selection can include assessing the amount or activity of brown adipose tissue in the subject and recording these observations. The evaluation can be performed before, during, and/or after the delivery of cold slurry. For example, the evaluation can be performed at least 1 day, 2 days, 4, 7, 14, 21, 30 or more days before and/or after the delivery of cold slurry.

The treatment methods can include assessing the treatment. For example, the amount or activity of brown adipose tissue in the subject following treatment is observed and recorded. This post-treatment observation can be compared to the observations made during subject selection. In some instances, the subject will have increased brown adipose tissue levels and/or activity. In other instances, the subject will show reduced symptoms.

The treatment assessment can include determining the subject's weight or BMI before and/or after treatment, and comparing the subject's weight or BMI before treatment to the weight or BMI after treatment. An indication of success would be an observation of a decrease in weight or BMI. In some examples, the treatment is administered one or more additional times until a target weight or BMI is achieved. Alternatively, measurements of girth can be used, e.g., waist, chest, hip, thigh, or arm circumference.

The treatment assessment can be used to determine the future course of treatment for the subject. For example, treatment may be continued without change, continued with change (e.g., additional treatment or more aggressive treatment), or treatment can be stopped. The treatment methods can include one or more additional deliveries of cold slurry, e.g., to increase non-shivering thermogenesis to maintain or further reduce obesity in the subject.

Excess fat poses a host of local and systemic problems, including increased risk for cardiovascular disease, type II diabetes, and cancer, associated particularly with excess visceral fat, and secondary problems due to being overweight including musculoskeletal problems, arthritis, and difficulty exercising. There are hints that adipose tissue is preferentially sensitive to cold injury. A rare clinical entity of cold-induced fat necrosis in infants, has been well described and sometimes called “popsicle panniculitis”. Inflammation of the buccal fatty tissue occurs after infants suck for a prolonged time on frozen treats. Another uncommon clinical entity, equestrian panniculitis, was described in females after horse riding with tight pants in cold climates. These unusual clinical observations suggest that human adipose tissue may be preferentially damaged by exposure to cold.

Based on the premise that fat cells are more easily damaged by cooling than skin cells, cryolipolysis was developed as a nonsurgical way to destroy fat cells. Cold is applied to a region of lipid-rich tissue (fat), effectively crystallizing adipose cells and inducing apoptosis, a natural cell death. Furthermore, localized panniculitis or inflammation of the tissue occurs later on; this leads to further removal of adipocytes (fat cells) as a result of phagocytosis. The loss of adipose tissue can continue after the cold application for 4 to 6 weeks.

In addition to reducing adipose tissue, cryolipolysis can also be used to reduce epicardial fat, pericardial fat, and visceral fat as described in U.S. application Ser. No. 13/574,425 and International Application No. PCT/US2015/047292, which are incorporated herein in their entireties. Other applications of cryolipolysis include treating obstructive sleep apnea, spinal cord lipomas, and lipomyelomeningocele, are also described in the aforementioned application.

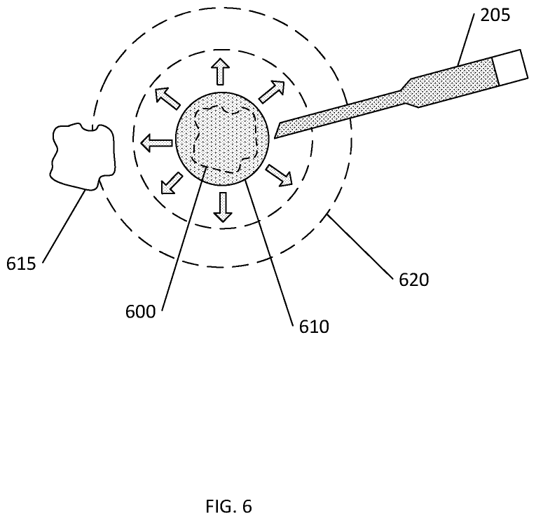

The cold slurry therapies of the present invention also include delivering a cold slurry to a nerve to mediate the neurological action of the nerve or to remove the nerve. FIG. 6 shows a nerve 600 (in phantom line) located inside a subject. The nerve 600 can be the vagus nerve, for example. Cold slurry can be delivered at or near the nerve 600 from a delivery device 205 that is located outside the subject's body. (The delivery device 205 and cold slurry are described in greater detail at the end this disclosure).

The cold slurry, shown in the figure as delivered cold slurry 610, can limit innervation or conduction of the nerve. Alternatively, the cooling effect of the delivered cold slurry 610 can cause the nerve 600 to undergo cell death, thereby, reducing the size of the nerve 600 or removing it all together. Either mechanism can be used to manage pain and/or to treat a neurological disorder, for example. After delivery, an affected area 620 expands to a size larger than the initial delivery site (shown in the figure as arrows radiating outwardly from the delivered cold slurry 610 and dashed circles of increasing size). The affected area 620 reaches a size encompassing a portion of an adjacent tissue 615.

The cooling effect of the delivered cold slurry 610 is localized to tissue being treated (i.e., the nerve 600) and surrounding tissue (i.e., the adjacent tissue 615). In this way, any discomfort caused by the cold therapy is limited. The cold slurry is sterile and biocompatible; and, as such, the delivered cold slurry 610 can be advantageously left in the body (e.g. no removal of the slurry is necessary after cooling has been effected).

Wallerian degeneration, the main form of axonal degeneration, can be a noted mechanism of action. It refers to the changes occurring in the distal segment of a peripheral nerve, particularly axonal degeneration and it's covering of the myelin sheath. The delivered cold slurry 610 can temporarily denervate or limit conduction of the nerve via axonal degeneration at and distal to the treatment site, while the acellular nerve structure remains intact, and the basal laminae of the endoneurium unaffected. Due to the preservation of the surrounding endoneural, perineural, and epineural structure, gradual axonal regeneration and re-myelination to normal levels is noted between several weeks and months.

Myelin, the fatty white substance surrounding axons of nerve cells, forms an electrically insulating layer and is essential for the proper functioning of nerve pathways. Because myelin's dry mass is approximately 70-85% lipid, these cells may crystallize as a result of cold slurry delivery, and undergo apoptosis and subsequent cell removal from an axon, which can remain intact. This mechanism can mimic that of what is seen in subcutaneous adipose tissue, however, re-myelination (production of myelin) in the treated nerve is assumed to occur in approximately 6 weeks along with restored functioning of neural pathways.

The delivered cold slurry 610 can also create vascular damage to the vasonervorum, the vessels supplying blood to nerve 600, which produces severe endoneural edema. The delivered cool slurry 610 would disrupt the nerve structure and create wallerian degeneration, but leave the myelin sheath and endoneurium intact. Additionally, local edema is decreased due to the decrease in vascular permeability that in turn reduces release of inflammatory mediators.

The reduction of neurologic innervation applies to both sensory and motor fibers, and in turn maintains applicability in sensory as well as motor disorders. A number of other neurological disorders can also be treated by delivering a cold slurry at or near the nerve, as described in International Application No. PCT/US2015/047292, which is incorporated herein in its entirety.

About 3.2 million Americans suffer from “chronic migraine”, which can be defined a distinct and severe neurological disorder characterized by patients who have a history of migraine and suffer from headaches on fifteen or more days per month with headaches lasting four hours a day or longer. A procedure for delivering a cold slurry to internal tissue, such as the procedure described above with reference with FIG. 6, can be used to treat chronic migraine. The procedure can include injecting a cold slurry at or near muscles located at multiple points (e.g., seven) around a patient's head and neck (e.g., using the delivery device 205 for FIG. 1). The contraction of these muscles is thought to be a trigger for a migraine. The cooling effect from the cold slurry delivered at or near the muscles causes the muscles to relax, which in turn, can alleviate the migraine.

Cold slurry can be used to reduce or eliminate symptoms associated with pain disorders caused by peripheral neuropathy, which may be associated with metabolic nerve damage, infection, trauma, genetic factors, and/or chemical processes. For example, cold slurry can be used to reduce pain in patients having chemotherapy-induced peripheral neuropathy or paclitaxel-induced acute pain syndrome.

Spasticity is a muscle control disorder that is characterized by tight or stiff muscles and an inability to control those muscles. In addition, reflexes may persist for too long and may be too strong (hyperactive reflexes). Spasticity is caused by an imbalance of signals from the central nervous system (brain and spinal cord) to the muscles. This imbalance is often found in people with cerebral palsy, traumatic brain injury, stroke, multiple sclerosis, and spinal cord injury. A variety of cold slurry delivery procedures can be used to reduce or inhibit muscle spasticity.

For example, an amount of cold slurry can be delivered internally so as to provide a cooling effect on components of the sensorimotor complex, including afferent fibers of muscle spindles, skin receptors, extrafusal muscle fibers, and the neuromuscular junction. Cooling these components has been found to reduce spasticity. In another example, an amount of cold slurry can be delivered at or near cold fibers (i.e., myelinated fibers for sensing cold stimuli). The sympathetic stimulation by these cold fibers produces vasoconstriction and decreases muscle spindle sensitivity, which in turn reduces spasticity. In yet another example, the delivery of cold slurry to an internal tissue can be used to change membrane polarization. Cooling a sensory terminal causes the terminal to become hyperpolarized, which in turn decreases discharge activity of a muscle spindle and reduces spasticity.

Cold slurry can be used to treat detrusor contractions or bladder spasms, which cause urgency incontinence (overactive bladder) and stress incontinence. The cooling effect of cold slurry delivered at or near the lumbosacral spinal cord can moderate the parasympathetic innervation to the bladder. This in turn suppresses bladder instability and simultaneously closes the urethral outlet.

Cold slurry can be used to treat hemifacial spasms, which cause facial twitches, tics, and convulsions. The cooling effect of cold slurry delivered at or near the seventh cranial nerve or facial nerve can limit motor innervation, which in turn can limit hemifacial spasms.

Cold slurry can be used to treat laryngospasms, which can temporarily make it difficult to speak or breathe. The cooling effect of cold slurry delivered at or near the vagus nerve can limit motor innervation, which in turn can limit laryngospasms.

A procedure for delivering an amount of cold slurry at or near internal tissues can be used to facilitate muscular contraction for various forms of neurogenic weakness and for muscle re-education. The cooling effect of cold slurry delivered at or near a muscle relaxes muscle spasms and minimizes upper motor neuron spasticity. This, in turn, allows for proper healing to occur without spasticity hindering the healing process.

Cold slurry can be used as therapeutic agent for treating hyperhidrosis/gustatory hyperhidrosis. Cold slurry can be used as an analgesic supplement to other hyperhidrosis treatments. For example, cold slurry can be used as cryoanalgesia to lessen the intensity of pain during injection of Botulinum toxin in patients with focal axillary hyperhidrosis. Cold slurry can also be used to destroy or inhibit apocrine and/or eccrine sweat glands as an intervention for hyperhidrosis.

A procedure for delivering an amount of cold slurry at or near internal tissues can be used to reduce an acute inflammatory response, for example after surgery. Surgery is inherently injurious to tissue and skin, and activates the body's natural response to stress and trauma. Inflammation is the body's attempt to heal—red blood cells initiate the inflammatory process and white blood cells accumulate to fight potential infection. Inflammation is necessary to heal in the short term, but longer term inflammation can be injurious and may slow down the healing process.

The cooling effect of delivering cold slurry at or near injured tissue lowers the temperature and metabolic rate of the injured tissue, and constricts blood vessels and blood flow. This promotes healing and inhibits inflammation. Once the cold slurry is removed (e.g., melts away), highly oxygenated, nutrient-rich blood flows to the injured area reducing pain, bruising and swelling. Advantageously, the cold slurry delivery procedure can accelerate surgical recovery, and reduces bruising and the formation of scar tissue.

Cold slurry can be used to treat restricted knee flexion caused by a traumatic lower extremity fracture. Rehabilitation after such a fracture typically involves range of motion exercises to address the loss of knee flexion or extension caused by the fracture. Often a patient undergoing the rehabilitation is in great pain when performing these exercises. To help the patient cope with the pain, an amount of cold slurry can be delivered to internal tissues around the knee to suppress the pain and reduce joint effusion during the exercises. Advantageously, this cold slurry delivery procedure allows the patient to do the range of motion exercises with less pain, which can lead to a quicker recovery.

A procedure for delivering an amount of cold slurry at or near internal tissues can be used to manage pain after surgery. As such, a cold slurry therapy can be an alternative to using an epidural analgesia or narcotic painkiller, which undesirably can be habit forming. For example, an amount of cold slurry can be delivered at or near an incision site to act as local anesthetic. In a thoracotomy example, an amount of cold slurry can be delivered, intraoperatively, at or near intercostal nerves above and below incision points to relieve postoperative pain associated with the thoracotomy.

As described in the examples above, delivering an amount of cold slurry at or near an internal tissue(s) can be part of a procedure for treating an ailment. In some these examples, the cooling effect of the delivered cold slurry can have the added benefit of reducing post-operative pain associated with a procedure. For example, in a renal sympathetic denervation procedure to treat arrhythmias, an amount of cold slurry can be delivered (via an endovascular catheter) at or near nerves in the wall of the renal artery. The cooling effect of the delivered cold slurry causes the cell death of these nerves. This is an alternative to ablating the nerves with radiofrequency energy or ultrasound. Unlike radiofrequency or ultrasound ablation, however, the cooling effect of a cold slurry delivery procedure has the added benefit of reducing procedural pain and vascular complications.

Used as a cryotherapeutic agent, cold slurry can provide an effective therapeutic response to pathologies affecting afferent nerve pathways that may lead to itching, burning or pain sensation. Examples pathologies that can be treated by a cold slurry delivery procedure include but are not limited to notalgia paresthetica, trigeminal neuralgia, phantom limb pain, neuroma (Morton's neuroma), postherpetic neuralgia, occipital neuralgia, tension headaches, and vulvodynia. In some cold slurry delivery procedures used on sensory nerves, cold slurry is used for cryoneurolysis (also referred to as cryonueuroablation, cryoanalgesia, and cryoneuromodulation). The cooling effect of the cold slurry delivered at or near a sensory nerve reduces innervation or conduction of the nerve. For example, the cooling effect of delivering a cold slurry at or near the trigeminal nerve reduces the sensory innervation to the trigeminal nerve.

In other cold slurry delivery procedures used on sensory nerves, the cold slurry is used for cryoablation. The cooling effect of the cold slurry delivered at or near a sensory nerve destroys or damages the nerve. For example, to treat occipital neuralgia, the cold slurry is delivered at or near the occipital nerves where the cooling effect of the delivered cold slurry ablates the occipital nerves.

A variety of dermatological disorders can treated by delivery a cold slurry at or near the affected tissue. For example, the cooling effect of a cold slurry can reduce inflammation and pain associated with such disorders as lichen sclerosus, lichen planus, atopic dermatitis (eczema), psoriasis, and prurigo nodularis. Cold slurry can also be used to treat an irritation or itching/burning sensation of the skin mediated by sensory nerves, including scalp itch and vulvar itch. In these examples, the cold slurry acts as an analgesic inhibiting sensory neuronal activity on the dermis. Cold slurry can further be used to reduce and/or flatten a keloid lesion, hypertrophic scar, and other superficial scarring. For example, the cooling effect of a cold slurry can destroy or reduce cells making up the scar thereby reducing its bulk.

Treating with a cold slurry can be an alternative to conventional ways of treating dermatological disorders. For example, to treat eczema, a cold slurry can be a viable substitute to a topical anti-inflammatory. Cold slurry treatment can also be an adjunct treatment and used with another treatment to enhance the effectiveness of that treatment. For example, to treat lichen simplex chronicus, a cold slurry can delivered at or near a lesion while applying a corticosteroid to the lesion.

Cold slurry can also be used to treat chest pain associated with pleurisy, lung cancer, asthma, rib fracture, muscle strain, and shingles. Taking each source of pain in turn, the cooling effect of cold slurry delivered at or near tissues lining the throat and lungs can reduce inflammation of these tissues as well as the pain associated with pleurisy. The cooling effect reduces the pain signals sent to the central nervous system. To treat pain associated with lung cancer, cold slurry can be used as a palliative measure for malignant cases. The cooling effect of a cold slurry delivered at or near the lung can alleviate symptoms of dyspnea and hemoptysis.

To treat pain associated with asthma, the cooling effect of cold slurry delivered at or near bronchial tubes can cause short-term dilation of the bronchial tubes leading to an increase in forced expiration volume. Additionally, the cooling effect of the cold slurry can have an inhibitory effect on chronic-inflammatory processes in the bronchial mucosa, thereby alleviating pain. To treat pain associated with fracturing a rib, the cooling effect of a cold slurry delivered at or near the fracture can reduce the inflammatory response from the injury and, thereby, alleviate the pain.

To treat pain associated with straining a muscle, the cooling effect of a cold slurry delivered at or near the strained muscle can reduce the pain by slowing down nerve impulses, reducing inflammation due to constriction of local blood vessels, and accelerating healing due to reducing the metabolic rate in the cells. To treat pain associated with shingles, the cooling effect of a cold slurry delivered at or near the affected tissue can reduce pain and discomfort by inhibiting or reducing sensory nerve activity. The cold slurry treatment has the added benefits of increased energy due to the tendency of cold slurry to boost metabolic rates; fewer tendencies to develop headaches, fever, and chills; and reduced reliance on medication to control symptoms.

Cold slurry can be used to treat coccydynia or tailbone pain. The cooling effect of a cold slurry delivered at or near blood vessels in the tailbone area can cause the vasoconstriction of these vessels. This in turn can reduce pain, and control inflammation and edema associated with coccydynia.

Cold slurry can be used to treat lower back pain and pain associated with a herniated disc (with or without radiculopathy). This cold slurry therapy can be used with a spinal decompression therapy. The combination of therapies can provide a safe and appropriate herniated disc treatment. Other benefits can include a diminishing effect on pain and headaches as well as improvements in concentration.

Cold slurry can be used to treat pain associated with osteoarthritis or facet joint syndrome, including lumbosacral facet syndrome and lumbar facet joint syndrome. Delivering a cold slurry at or near a joint between two vertebrae in the spine can provide temporary pain relief. The cooling effect of the cold slurry can relax the surrounding muscles, thereby leading to a decrease in nociceptive information.

Delivery of a cold slurry to an internal tissue can also be used to treat a variety of diseases of the tongue. For example, hemangioma, a benign vascular tumor that can form in the subcutaneous layer of the tongue, can be treated by delivering an amount of cold slurry at or near the subcutaneous lesion. Leukoplakia, a predominantly white lesion of the oral mucosa, can be treated by delivering an amount of cold slurry at or near the lesion. A cold slurry delivery procedure can also be used to treat “burning mouth syndrome”, a chronic pain disorder commonly involving the tongue. In this procedure, an amount of cold slurry is delivered at or near the hypoglossal nerve and pharyngeal branch of the vagus nerve.

The delivery of a cold slurry to internal tissues can also be used in cryoamputation. The procedure involves applying an occlusive tourniquet to isolate an affected extremity of a patient and delivering a cold slurry to the affected internal tissues (e.g., using the delivery device 205 for FIG. 1). This cools the extremity and allows for the patient to be medically optimized prior to undergoing a formal amputation. For example, patients with gangrenous or necrotizing infections of the extremities causing hemodynamic instability can be resuscitated before amputation by using cryoamputation to isolate the infection from circulation.

The foregoing technique of delivering a cold slurry to internal tissues of an affected extremity allows a high-risk emergency amputation to be performed in an elective fashion after medical optimization. Advantageously, the patient can be adequately resuscitated, and amputation can be safely delayed until the patient is stable enough to tolerate un-rushed surgery.

Delivery of a cold slurry to an internal tissue can also be used to treat a variety of cancers. For example, primary cutaneous B-cell lymphoma, actinic keratosis (a pre-cancerous skin growth), squamous and basal cell carcinomas, and conjunctival lymphoma can be treated by delivering an amount of cold slurry at or near the respective lesion site. In some instances, the cold slurry delivery procedure can provide a better alternative to traditional methods. For example, cryotherapy to treat conjunctival lymphoma provides a lower cost alternative to radiation therapy with fewer ocular and systemic complications.

After being in accident like a car collision, an injured person may be suffering from soft tissue injuries, fractures, bleeding, and/or tearing of vital organs and blood vessels. Left untreated, these traumatic injuries can result in severe disability and death. A traumatic injury can be treated using a procedure for delivering a cold slurry at or near the injured tissue. Delivering the cold slurry at or near the injured area (e.g., using the delivery device 205 for FIG. 1) causes vasoconstriction and decreases local tissue temperature. In turn, vasoconstriction decreases blood flow to the injured area to limit hemorrhage, while the decrease in local cell metabolism prevents cell death. This cold slurry delivery procedure can also be used for other traumatic injuries, including blunt, penetrating, and thermal traumas. For example, the cold slurry delivery procedure can be used to reduce swelling/edema, reactive hyperemia or reduce muscle efficiency. The delivery of a cold slurry can also have an analgesic effect due to impaired neuromuscular transmission.

Cryotherapy utilizes the principle of inducing tissue destruction by freezing and thawing using, for example, argon and helium gasses, respectively. Ablative treatments are particularly useful in the elderly patients, those with comorbidities or in patients with small renal masses (SRMs) in solitary kidneys or renal impairment. Ablative therapies have less procedure-related complications and have promising medium-term oncological outcome. Longer-term results are accumulating. Cryotherapy may be a better modality for oncological control than radiofrequency ablation (RFA). Ablative therapy has emerged as a viable treatment option for SRMs with recurrence free survival rates approaching that of extirpative surgery.

Fibroadenomas are solid, noncancerous breast tumors that occur most often in adolescent girls and women under the age of thirty. Fibroadenomas are among the most common breast lumps in young women. Treatment may include monitoring to detect changes in the size or feel of the fibroadenoma, a biopsy to evaluate the lump, or surgery to remove it.

A procedure for delivering a cold slurry at or near a fibroadenoma is a minimally invasive, non-surgical alternative to treatments such as a lumpectomy. The procedure delivers the cold slurry at or near the fibroadenoma to destroy or reduce the size of the fibroadenoma. The procedure can include delivering the cold slurry (e.g., using the delivery device 205 for FIG. 1) under ultrasound guidance. The procedure can also include freezing the fibroadenoma with a first delivery of cold slurry, thawing the fibroadenoma, and then re-freezing the thawed fibroadenoma with a second delivery of cold slurry. This sequence of freezing, thawing, and freezing may help in destroying or reducing the size of the fibroadenoma. The foregoing procedure may also be used on breast tumors.

It is well known that hypothermia can postpone damage to tissues caused by inadequate blood supply and oxygen deprivation. One important example of the potential protective properties of hypothermia is in the area of cardiac arrest. The ability of cardiac cells to survive severe ischemia can be significantly enhanced by transient hypothermia. Application of cold slurry to cardiac, as well as other tissues can reduce tissue injury caused by the sudden reperfusion of ischemic tissue.

A cold slurry can be delivered via the gastrointestinal tract to cool organs adjacent to the gastrointestinal tract, including the lungs, heart, kidneys, gallbladder, and spleen. The human gastrointestinal tract can be divided into an upper gastrointestinal tract and a lower gastrointestinal tract. The upper gastrointestinal tract includes the mouth, esophagus, stomach, and duodenum. The beginning of the gastrointestinal tract, the mouth, defines a point of entry for a first cold slurry delivery procedure. In this procedure, an appropriately sized tube or catheter is inserted into the patient's mouth and advanced through the upper gastrointestinal tract until the tube/catheter reaches a desired location between the patient's month and duodenum. Once reached, an amount of cold slurry can be delivered (e.g., using the delivery device 205 of FIG. 1 coupled to the tube/catheter) thereby cooling the organ(s) adjacent to the delivery location.

The gastrointestinal tract includes the small intestine and large intestine, which in turn includes the colon, rectum, and anus. The end of the gastrointestinal tract, the anus defines a point of entry for a second cold slurry delivery procedure. In this procedure, an appropriately sized tube or catheter is inserted into the patient's anus and advanced through the lower gastrointestinal tract until the tube/catheter reaches a desired location between the patient's anus and duodenum. Once reached, an amount of cold slurry can be delivered (e.g., using the delivery device 205 of FIG. 1 coupled to the tube/catheter) thereby cooling organ(s) adjacent to the delivery location.

The choice of which of the cold slurry delivery procedures to use can be based on which organ is being targeted for cooling. Other factors, such as patient comfort, can also be considered when choosing which of the cold slurry delivery procedures to use. Delivering a cold slurry through the gastrointestinal tract, as described above, is advantageous because the gastrointestinal tract is a natural conduit through a patient's body that passes near many of organs. Some of these organs are difficult to reach from outside a patient (e.g., because of location and/or proximity to other organs). As such, the procedure for delivering a cold slurry by way of the gastrointestinal tract can be a convenient way of cooling the body cavity.

Atherosclerosis or the hardening of the arteries is caused by the buildup of plaque (fatty deposits, calcium deposits, and scar tissue) in the arteries. Left untreated, atherosclerosis can lead to a heart attack or stroke; which are the two leading causes of death and disability in the United States for both men and women. Treatment can include endovascular stent surgery in which a tiny wire mesh tube called an endovascular stent is placed in an affected artery to correct the narrowing of the artery blocked by plaque.

While placing the endovascular stent in the affected artery, there is a risk that some of the plaque may be dislodged from the artery walls and block the artery. Undesirably, this can result in a patient having a heart attack or stroke during an endovascular stent surgery. To protect against this risk, a procedure to delivery cold slurry to harden plague before placing a stent can be used. The procedure includes threaded a catheter with a deflated balloon at its tip through an incision in a patient's groin up into an affected blood vessel. The entire procedure can viewed with a fluoroscope. The balloon is then filled with a cold slurry thereby enlarging the balloon causing it to contact the plaque on the blood vessel walls. The delivered cold slurry, in turn, cools and hardens the plaque.

Once the plaque is cooled and hardened, the balloon is deflated (e.g., melting or removing the cold slurry) and the catheter is removed from the patient. The endovascular stent is then threaded through the same incision into the affected blood vessel on another catheter with a deflated balloon at its tip and inside the stent. The balloon catheter is guided to the blocked area and the balloon is inflated, causing the stent to expand and press against the cooled and hardened plaque on the vessel walls. The balloon is then deflated and taken out of the vessel. The stent remains in the vessel permanently to hold the vessel walls open and allow blood to pass freely as in a normally functioning healthy artery. Cells and tissue will begin to grow over the stent until its inner surface is covered. It then becomes a permanent part of the functioning artery.

In a convenient example of the procedure, a single catheter can be used to deliver the cold slurry and place the endovascular stent. Advantageously, such an example can reduce the amount of time it takes to perform the procedure.

As previously described, the delivery device 205 for FIG. 1 can be used to deliver cold slurry to internal tissue. In more detail, the delivery device 205 is capable of providing continued agitation to the cold slurry at the point of care, such as through rotation of blades within the delivery device 205, use of vibration, or both. The cold slurry can be cooled/kept cool inside the delivery device 205 through the use of a small profile cooling sleeve that easily slips over the delivery device 205 and provides cooling at the point of care. The cooling sleeve can cool or maintain the temperature of the cold slurry through a number of mechanisms, such as the provision of a refrigerant, the triggering of an endothermic reaction, and the compression of gas. Other examples of the delivery device are described in U.S. Provisional Application No. 62/300,679 filed on Feb. 26, 2016 and U.S. Provisional Application No. 62/416,484 filed on Nov. 2, 2016, which are incorporated herein in their entireties.

The cold slurry can be made from any sterile, biocompatible fluid that is capable of being cooled to provide a cold slurry. The cold slurry can be generated in the delivery device 205 itself by providing the fluid to the delivery device 205 and cooling the fluid within the delivery device 205 while agitating the fluid. The cold slurry can also be produced in a separate chamber and then transferred to the delivery device 205. Other examples of devices for making cold slurry and methods for making cold slurry are also described in U.S. Provisional Application No. 62/416,484.

Preferably, the temperature of the fluid is cooled to or below about 10° C., 7° C., 5° C., 4° C., 3° C., 2° C., 1° C., 0° C., −1° C., −2° C., −3° C., −4° C., −5° C., −10° C., −15° C., −20° C., −30° C., −40° C., and −50° C. The cold slurry generated has a plurality of sterile ice particles and is suitable for delivery into a subject. Example slurry compositions, slurry temperatures, and cross-sectional dimensions of ice particles are provided in U.S. Provisional Application No. 62/300,679; and International Application Nos. PCT/US2015/047292 and PCT/US2015/047301, which are incorporated herein in their entireties. It is to be understood that an advantage of the cold slurry in accordance with the present invention is that the composition of the cold slurry is suitable to delivery to tissues within the body, such that the slurry can be delivered to a tissue within the body of a patient and remain within the body (e.g. no removal of the slurry is necessary after cooling has been effected).

What is claimed is:

1. A method for inducing non-shivering thermogenesis in brown adipose tissue in a subject, the method comprising:
delivering an effective amount of cold slurry to tissue that is internal to a subject.

2. The method of claim 1, wherein the internal tissue to which the cold slurry is delivered is adjacent to tissue including a cold thermoreceptor.

3. The method of claim 1, wherein the internal tissue to which the cold slurry is delivered includes a cold thermoreceptor.

4. The method of claim 3, wherein the internal tissue with the cold thermoreceptor is any one of: adipose tissue, colonic tissue, abdominal tissue, and hypothalamic tissue.

5. The method of claim 1, wherein delivering includes injecting the cold slurry using a syringe.

6. The method of claim 1, wherein delivering includes delivering the cold slurry through a catheter.

7. The method of claim 1 further comprising making the cold slurry in a syringe used to deliver the cold slurry.

8. The method of claim 1 further comprises administrating the effective amount of cold slurry to treat obesity or a weight-related disorder.

9. The method of claim 8 further comprising selecting the subject to whom to administer the cold slurry.

10. The method of claim 8 further comprising assessing the result of administrating the cold slurry.

11. A method comprising:
delivering an effective amount of cold slurry to a nerve selected from group consisting of peripheral nerve, autonomic nerve, somatic nerve, parasympathetic nerve, sympathetic nerve, interneurons, cervical plexus nerve, brachial plexus nerve, lumbar plexus nerve, sacral plexus nerve, coccygeal plexus nerve, celiac plexus nerve, Aurbach's plexus nerve, Meissner's plexus nerve, lesser occipital nerve, greater auricular nerve, transverse cervical nerve, ansa cervicalis, supraclavicular nerve, phrenic nerve, musculocutaneous nerve, axillary nerve, radial nerve, median nerve, ulnar nerve, iliohypogastric nerve, ilioinguinal nerve, genitofemoral nerve, lateral cutaneous nerve, femoral nerve, obturator nerve, superior gluteal nerve, inferior gluteal nerve, posterior cutaneous nerve, tibial nerve, peroneal (Fibular) nerve, pudendal nerve, sciatic nerve, pedestal nerve, lower intercostal nerve (transverse abdomens nerve), paravertebral nerve, infraclavicular nerve, intercostal nerve, interscalene nerve, trigeminal nerve, cutaneous nerve, spinal nerve, cranial nerves, motor nerves, sensory nerve, enteric nerve, subcutaneous nerve.

12. A method comprising:
delivering an effective amount of cold slurry to a perineurial sheath of a peripheral nerve.
